(19) United States
(12) Patent Application Publication
(10) Pub. No.: US 2002/0055452 A1
(43) Pub. Date: May 9, 2002

(54) Process for imparting a fragrance to a product and fragrance and conditioning to a dry fabric

(75) Inventors: Thomas McGee, Nanuet, NY (US); Richard P. Sgaramella, Hoboken, NJ (US); Stuart Bernard Fraser, Cheshire (GB)

Correspondence Address: Kevin C. Hooper, Esq., Bryan Cave, LLP, 245 Park Avenue, New York, NY, 10167-0034, US

(73) Assignee: Givaudan SA

(21) Appl. No.: 09/932,511

(22) Filed: Aug. 17, 2001

Related U.S. Application Data
(63) Continuation-in-part of application No. PCT/US01/21334, filed on Jul. 5, 2001.
(60) Provisional application No. 60/216,571, filed on Jul. 7, 2000.

(51) Int. Cl. A61K 7/46

(52) U.S. Cl. 512/2; 512/3

(57) ABSTRACT

The present invention provides a fragrance delivery vehicle including a water insoluble oil, a conditioner containing a cationic active at a level above about 0.5%(wt), and at least about 0.1%(wt) of a fragrance composition comprising a first aroma chemical with a clogP of 3.0 or less and a second aroma chemical having a clogP of at least 4.0. Processes for conditioning and fragrancing a substrate, for enhancing the fragrance of a consumer product, and for designing a fragrance delivery vehicle are also provided.

### FIELD OF THE INVENTION

The present invention relates to a fragrance delivery vehicles and processes for imparting optimal fragrance perception to a product and to a substrate, such as a dry fabric or hair. More particularly, the invention relates to a fragrance delivery vehicle that includes a water insoluble oil, a conditioner containing a cationic active at a level above about 0.5%(wt), and at least about 0.1%(wt) of a fragrance composition containing a first aroma chemical with a clogP of 3.0 or less and a second aroma chemical having a clogP of at least 4.0. Processes for conditioning and fragrancing a substrate and fragrancing a product and for designing a fragrance delivery vehicle are also provided.

### BACKGROUND OF THE INVENTION

Liquid conditioning products for treating substrates, such as hair and fabric conditioners, contain cationic materials as key active ingredients. Other ingredients, such as oils as disclosed in WO 97/44424, may be used in liquid conditioning products together with a cationic active to aid in the deposition of fabric treatment agents. (See also WO98/16538, which is incorporated by reference as if recited in full herein.). Such oils, as part of the conditioner, may be delivered during the final rinse.

It is known that the composition of a fragrance for a liquid conditioner must provide fragrance in sufficient quantities to be perceived in the product and during use on damp and dry substrates. The principles of achieving substantivity and longevity on treated substrates are disclosed by Muller et al., “What Makes A Fragrance Substantive,” Perfumer and Flavorist, 1845-1849 (1993) and Escher et al., “A Quantitative Study of Factors that Influence the Substantivity of Fragrance Chemicals on Laundered and Dried Fabrics,” JOACS, vol. 71, no. 1 (1994).

Muller discloses that the greater the quantity of water insoluble aroma chemicals with low vapor pressures in the fragrance composition, the greater the substantivity of the fragrance. The solubility of an aroma chemical is conventionally measured using a number of different procedures, such as for example the procedure disclosed in Etsweiler et al., Analytical Chemistry 67; 655-658 (1995). Low solubility is highly correlated with the hydrophobicity of an aroma chemical.

Escher uses hydrophobicity, determined as the partition coefficient of the chemical between octanol and water and expressed as log P. Log P may be measured directly but more conveniently, it may be calculated from the structure of the molecule, using one of several commercially available software programs, such as ACD Software, ACD/logP calculator version 4.0, Advanced Chemistry Development, Toronto, Ontario Canada. The calculated value is abbreviated to clogP.

The vapor pressure of an aroma chemical, which is related to its boiling point, is also considered to ensure that it does not evaporate during the rinse cycle to which the conditioner is added, and that it survives the drying process ensuring that sufficient aroma chemical is present to provide a lasting scent on the dry substrate. High boiling point aroma chemicals will evaporate more slowly giving a longer lasting fragrance perception. Vapor pressure and boiling point may be measured or calculated using one of the commercially available software programs, such as ACD Software, ACD/Boiling Point calculator version 4.0.

Another way to impart a fragrance to a substrate is to have the substantive part of the fragrance composed of an aroma chemical with low perception thresholds. The lower the perception threshold of an aroma chemical, the lower the quantity that is required to smell it. A variety of techniques are available to determine the perception threshold. See, e.g., Neuener-Jehle and Etzweiler in Art Science and Technology, editors Lampaski and Muller, ch. 6, 153-212 (1991).

In sum, it is known that a fabric softener perfume that provides a long lasting fragrance will contain a significant proportion of low solubility (higher clogP) and high boiling point (low vapor pressure) fragrance chemicals, preferably with low perception thresholds. Bacon et al., U.S. Pat. No. 5,652,206 (“Bacon”) defines, for fabric softeners, a minimum quantity of fragrance materials with high clogP and high boiling point. Bacon discloses that the softener must contain at least 0.01% to about 10% of an enduring perfume containing at least 70%(wt) of enduring perfume ingredients selected from ingredients having a boiling point of at least about 250° C. at 760 mm Hg and a clogP of at least about 3. Bacon also discloses that non-enduring perfume ingredients are to be minimized in such softener compositions.

Bacon distinguishes between enduring and non-enduring compositions and specifically identifies the following compounds as not non-enduring: cis-jasmone, dimethyl benzyl carbinyl acetate, ethyl vanillin, geranyl acetate, alpha-ionone, beta-ionone, gamma-ionone, koavone, lauric aldehyde, methyl dihydrojasmonate, methyl nonyl acetaldehyde, gamma-nonalactone, phenoxy ethyl iso-butyrate, phenyl ethyl dimethyl carbinol, phenyl ethyl dimethyl carbinyl acetate, alpha-methyl-4-(2-methylpropyl)-benzenepropanal, 6-acetyl-1,1,3,4,4,6-hexamethyl tetrahydronaphthalene, undecylenic aldehyde, vanillin, 2,5,5-trimethyl-2-pentylcyclopentanone, 2-tert-butylcyclohexanol, verdox, para-tert-butyleyclohexyl acetate, or ingredients having a boiling point of less than about 250° C., or having a clogp of less than about 3.0, or having both a boiling point of less than about 250° C. and a clogp of less than about 3.0.

Bacon recognizes that in certain fabric softener compositions, some non-enduring perfume ingredients may be used in small amounts, e.g., to improve product odor. To minimize waste and pollution, however, the enduring perfume compositions of Bacon contain less than about 30%(wt) of non-enduring perfume ingredients. In more preferred embodiments, the enduring perfume compositions of Bacon contain less than 25, 20, and 15 wt. % of non-enduring perfume ingredients.

Examples of suitable non-enduring fragrances with a clogp of less than 3 and a boiling point less than 250° C. are benzaldehyde, benzyl acetate, laevo carvone, geraniol, hydroxycitronellal, linalool nerol, phenyl ethyl alcohol and alpha terpineol. Examples of non-enduring perfume with a boiling point >250° C. and a clogp <3.0 are coumarin, eugenol, iso eugenol, indole, methyl cinnamate, methyl-n-methyl anthranilate, and beta methyl naphthyl ketone. Examples of non-enduring fragrance ingredients with a boiling point <250° C. and clogp>3.0 are iso-Bomyl acetate, carvacrol, alpha-citronellol paracymene, dihydro myrcenol, d-limonene, and linalyl acetate.

Trihn, et al. U.S. Pat. No. 5,833,999 uses the same criteria as Bacon to define enduring fragrances for hair and skin products.

The active agents disclosed as conditioners by, e.g. Bacon and Trihn, are predominantly cationic, having at least one of the alkyl chains with a chain length at least equal to 12, preferably greater than 16. The level of cationic active used in the conditioner product depends upon whether the product is dilute or concentrated. The levels of cationic active in the dilute products range from 1%(wt) to 7%(wt) and in concentrated products from 10%(wt) to 20%(wt).

In summary, optimum fragrancing of both product and dry substrates has heretofore been difficult to achieve using the methods summarized above, which rely solely on water insolubility, boiling point, and perception threshold. Using these conventional approaches, limitations are placed on the aroma chemicals available for the perfumer to create good hedonic fragrances.

### SUMMARY OF THE INVENTION

In the present invention, it has been found that enhanced deposition of fragrance onto a treated substrate is achieved with a cationic active and a water-insoluble oil. This gives enhanced fragrance perception on dry fabric without the need to have at least 70%(wt) of the fragrance with clogP greater than 3 and a boiling point greater than 250° C. However, in order to maintain at least as good a perception of fragrance from the product compared to the perception of fragrance that is achieved from a simple cationic conditioning vehicle, one has to formulate the fragrance specifically for the delivery vehicle.

Surprisingly, it has been found that a water insoluble oil, when combined with a cationic active, provides excellent fragrance deposition and perception to dry substrates, such as fabrics or hair, without necessitating high levels of fragrance chemicals with a clogP of 3 or higher and a boiling point of greater than 250° C. Using such a composition, good fragrance perception is also provided to the product so long as the fragrance composition is constructed according to the present invention.

In particular, the inclusion of an oil in, e.g. a fragrance delivery vehicle, substantially enhances the deposition of a substantive aroma chemical, i.e. one that has a clogP higher than 3.0. These oils may be present in dilute products from about 0.5%(wt) to about 7%(wt) and in concentrated products from about 1%(wt) to about 20%(wt).

The inclusion of such an oil, however, depresses the fragrance of the product. To offset this would require a large increase in fragrance materials with the higher clogP values and high boiling points, such as the levels summarized above. In the present process, however, it is now possible to maintain an enhanced perception of the fragrance on, e.g., a dry fabric, and to maintain at least an equal perception in the product, if the composition of the fragrance is constructed as set forth in more detail below.

Accordingly, one embodiment of the present invention is a fragrance delivery vehicle comprising a water insoluble oil, a conditioner containing a cationic active at a level above about 0.5%(wt), and at least about 0.1%(wt) of a fragrance composition comprising a first aroma chemical with a clogp of 3.0 or less and a second aroma chemical having a clogP of at least 4.0.

Another embodiment of the present invention is a process for conditioning and fragrancing a substrate and fragrancing a product comprising combining a fragrance delivery vehicle with a product for conditioning and fragrancing a substrate, the fragrance delivery vehicle comprising a water insoluble oil, a conditioner containing a cationic active at a level above about 0.5%(wt), and at least about 0.1%(wt) of a fragrance composition comprising a first aroma chemical with a clogp of 3.0 or less and a second aroma chemical having a clogP of at least 4.0; and contacting the product containing the fragrance delivery vehicle with a substrate in an aqueous medium.

A further embodiment of the present invention is a process for enhancing the fragrance of a consumer product comprising incorporating a fragrance improving quantity of a fragrance delivery vehicle according to the present invention into a consumer product.

A further embodiment of the present invention is a process for designing a fragrance delivery vehicle that provides fragrance to a consumer product and that provides fragrance and conditioning to a dry substrate, which process comprises selecting a first aroma chemical having a clogP of 3 or less, which first aroma chemical is further selected from the group consisting of aroma chemicals having a vapor pressure at 25° C. of greater than 0.07 mm Hg, aroma chemicals having a threshold value at 25° C. of less than 100, and aroma chemicals having a vapor pressure at 25° C. of at least 0.07 mm Hg and a threshold value at 25° C. of less than 100; combining the first aroma chemical with a second aroma chemical having a clogP of at least 4.0 to form a fragrance composition; and incorporating at least about 0.1%(wt) of the fragrance composition with a water insoluble oil and a conditioner containing a cationic active at a level above about 0.5%(wt) to form the fragrance delivery vehicle.

### BRIEF DESCRIPTION OF THE DRAWINGS

FIG. 1 is a graph showing the % Reduction in Fragrance using an oil based fragrance delivery vehicle according to the present invention compared to a conventional non-oil based delivery vehicle. In FIG. 1, % Reduction in Fragrance is calculated as follows:

$$\% Reduction in Fragrance = \frac{( HS HEQ - HS HEQ / SPE ) \times 100}{HS HEQ}$$

Where HS is headspace and HEQ and SPE are defined in Example 1.

### DETAILED DESCRIPTION OF THE INVENTION

As noted above, a problem solved by the present invention is the design of a formulation for adding or providing a fragrance to a consumer product and/or to a substrate treated with the consumer product. In the present invention, such a formulation is referred to as a “fragrance delivery vehicle.” As also noted above, one problem with conventional fragrance compositions that use a non-oil based fragrance delivery vehicle to impart a fragrance to a consumer product and/or to a substrate treated with such a consumer product, is that large quantities (e.g. 70%(wt)) of high clogP value aroma chemicals are required to achieve adequate fragrancing. This requirement is disadvantageous because it necessitates the use of increased amounts of the aroma chemicals, which is commercially impractical. Moreover, the use of large quantities of aroma chemicals having high clogP values (i.e. above 3.0 or 4.0) limits the perfumer's ability to blend aroma chemicals into pleasing fragrances because there is a limit to the total amount of a fragrance composition that may be incorporated into standard fragrance delivery vehicles.

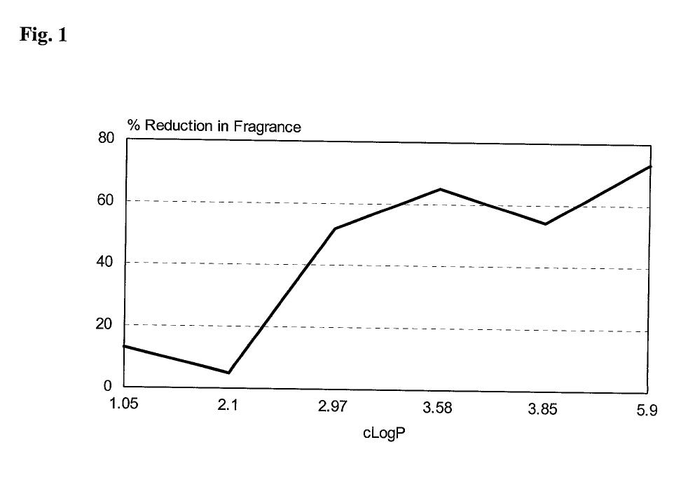

In the present invention, these and other disadvantages have been overcome by the use of an oil-based fragrance delivery vehicle. The use of such an oil-based vehicle for delivering fragrance compositions to products and substrates was conventionally believed to be commercially impractical because the oil in such vehicles causes a large % reduction in fragrance compared to non-oil based vehicles using the same aroma chemicals. As FIG. 1 shows, there is approximately a 60% reduction in fragrance in an oil-based fragrance delivery vehicle (HEQ/SPE) compared to a conventional non-oil based fragrance delivery vehicle (HEQ).

Unexpectedly, however, it is demonstrated here that aroma chemicals with a clogp of 3 or less have a significantly lower % reduction in fragrance in the oil-based fragrance delivery vehicles of the present invention. Accordingly, it is now possible to select aroma chemicals with clogP values of 3 or less that have a % reduction in fragrance of less than 60% and to further reduce the % reduction in fragrance by further selecting aroma chemicals that not only have a clogp of 3 or less but that have high vapor pressures and/or low threshold values.

Thus, by selecting aroma chemicals with a clogP of 3 or less and high vapor pressures and/or low threshold values, a fragrance composition may be designed that contains relatively small amounts of such aroma chemicals. This is beneficial to the perfumer because it reduces the cost of the fragrance composition, because smaller quantities of aroma chemicals are required, and it provides flexibility to the perfumer to be able to design mixtures of aroma chemicals that provide pleasing fragrances to product and/or substrates treated with the product.

Accordingly, the fragrance delivery vehicle of the present invention contains a water insoluble oil, a conditioner containing a cationic active at a level above about 0.5%(wt), and at least about 0. 1%(wt) of a fragrance composition based on the total weight of the fragrance delivery vehicle. The fragrance composition contains a first aroma chemical with a clogp of 3.0 and a second aroma chemical having a clogP of at least 4.0.

Preferably, the first aroma chemical has a clogp of 2.5 or less or a clogP that provides a % reduction in fragrance of less than 40%, preferably less than 20%, for example less than 10% using the method set forth in FIG. 1.

In the present invention, the first aroma chemical may be present in the fragrance composition as a mixture of aroma chemicals having a clogP of 3.0 or less. Likewise, the second aroma chemical may be present in the fragrance composition as a mixture of aroma chemicals having a clogP of at least 4.0. Preferably, the fragrance composition contains a mixture of both aroma chemicals having a clogP of 3.0 or less and aroma chemicals having a clogP of at least 4.

Preferably, the first aroma chemical is present in the fragrance delivery vehicle in a quantity that is at least about 0.5%(wt), more preferably at least about 1.0%(wt) based on the total weight of the fragrance delivery composition.

Preferably, the first aroma chemical (i.e. the aroma chemical with a clogP of 3.0 or less) has a vapor pressure at 25° C. of greater than about 0.07 mm Hg, more preferably greater than about 0.7 mm Hg, such as for example, greater than about 1.0 mm Hg. Thus, the first aroma chemical may have a vapor pressure of between about 0.07 mm Hg and about 20.0 mm Hg. Table 1 below provides exemplary aroma chemicals having a clogP of 3.0 or less that may be used by a perfumer in formulating a fragrance delivery vehicle according to the present invention. The vapor pressures (VP) of these aroma chemicals, as well as the clogP and threshold (TH) values, are thus within the scope of the present invention.

TABLE 1

| | | VP (mm | | | |
|---|---|---|---|---|---|
| | | Hg @ | | | |
| | clogP | 25° C.) | TH | Intensity | Level |
| Phenyl ethyl alcohol | 1.4 | 0.074 | 3 | 87 | 5-10 |
| Phenyl ethyl formate | 1.96 | 0.08 | 5 | 135 | |
| Fructone | 0.58 | 0.086 | 4.6 | 175 | |
| Methyl phenyl | 0.58 | 0.086 | 0.9 | 792 | |
| acetate | | | | | |
| Methyl Amyl | 2.0 | 4.732 | 85 | 239 | |
| Ketone | | | | | |
| Methyl Hexyl | 2.5 | 1.725 | 20 | 360 | |
| Ketone | | | | | |
| Ethyl phenyl acetate | 2.5 | 0.105 | 1.3 | 371 | |
| Cyclal | 2.67 | 0.578 | 8 | 585 | |
| cis-3hexenyl formate | 2.1 | 2.567 | 57 | 458 | |
| me phen ethy; oxide | 2.1 | 1.788 | 14.7 | 287 | |
| carvone laevo | 2.2 | 0.066 | 0.9 | 644 | 1-2 |
| Methyl phenyl | 2.0 | 0.129 | 0.9 | 810 | |
| acetate | | | | | |
| Prenyl acetate | 2.1 | 3.987 | 17.7 | 877 | |
| isobutyl acetate | 1.6 | 18.05 | 94 | 979 | |
| octenol jd | 2.6 | 0.531 | 2.5 | 932 | |
| para cresyl acetate | 2.0 | 0.176 | 0.7 | 1270 | |
| Cyclohexyl acetate | 2.2 | 0.978 | 7.2 | 1292 | |
| para tolyl aldehyde | 2.1 | 0.263 | 1.5 | 1440 | |
| Cis-3-hexenol | 1.61 | 1.039 | 13 | 2451 | |
| Aldehyde c7 | 2.5 | 3.854 | 5.3 | 3719 | 0.5-1.0 |
| Aldehyde c8 | 3.0 | 1.409 | 0.6 | 5938 | |
| ethyl caproate | 2.8 | 1.665 | 1.3 | 7453 | |
| et-2-me-butyrate | 1.6 | 7.853 | 7 | 6727 | |
| ethyl butyrate | 1.8 | 13.94 | 13.1 | 6793 | |
| Phenyl acetaldehyde | 1.8 | 0.368 | 0.1 | 20600 | 0.1-0.5 |
| Manzanate | 2.7 | 2.908 | 0.3 | 62586 | |

Preferably, the second aroma chemical (i.e. the aroma chemical having a clogP of at least 4.0) has a vapor pressure at 25° C. of less than about 0.02 mm Hg, preferably less than about 0.01 mm Hg, such as for example less than about 0.008 mm Hg. In the present invention, the clogP and vapor pressure were calculated using ACD/clogP calculator version 4.0 and ACD/Boiling Point calculator version 4.0, respectively, from Advanced Chemicals Development ACD/Labs Software (Toronto, Ontario, Canada).

The first aroma chemical in the fragrance composition preferably has a perception threshold below 100 ng per liter at 25° C., preferably, a perception threshold below 50 ng per liter at 25° C., such as for example, a perception threshold below 30 ng per liter at 25° C.

In the present invention, the fragrance composition contains at least about 1.0%(wt) of the first aroma chemical preferably at least about 5%(wt), such as for example from 10-20%(wt) based on the weight of the fragrance composition. Likewise, the fragrance composition contains at least about 20%(wt) of the second aroma chemical, such as about 30%, 40%, 50% or 60%(wt) based on the weight of the fragrance composition. The balance of the fragrance composition may contain additional fragrances or filler materials conventionally used by a perfumer.

In the present invention, the fragrance composition is present in the fragrance delivery vehicle at a level that is at least about 0.1%(wt), such as for example at least about 0.2%(wt) or at least about 0.3% based on the weight of the fragrance delivery vehicle. Preferably, the fragrance composition is between about 0.1% to about 2.0% (wt) based on the weight of the fragrance delivery vehicle.

As used herein, the phrase “fabric conditioner” means a product that imparts softness, drape, and other similar benefits, such as antistatic properties, color care properties, etc., to fabrics. As used herein the phrase “hair conditioner” means a product that imparts softness, easier combing, and shine to hair. In the present invention, the term “conditioner” is used throughout to refer to fabric conditioners, hair conditions, or both, as the context may dictate.

The phrase “aroma chemical” as used herein means a chemical that is volatile and that is detected by the nose as a smell. As noted above, the phrase “perception threshold” or “threshold” means the lowest quantity of an aroma chemical, in the vapor phase, that may be perceived by the nose.

In the present invention, the cationic active may be selected from dialky cationic actives, monoalky cationic actives, and mixtures thereof. The dialky cationic active in the conditioner of the fragrance delivery vehicle may be, for example, dialkyldimethyl ammonium chloride, dialkyldimethyl ammonium methyl sulfate, di (hydrogenated tallow) dimethyl ammonium chloride, dihexadecyldiethylammonium chloride, distearyldimethylammonium chloride, dibehenyldimethylammoinium chloride, di(coconut alkyl)dimethyl ammonium chloride, ditallowdimethyl ammonium chloride, ester quatemium compounds, dialkylyloxy dimethyl ammonium chloride, N,N-di(tallowyl-oxy-ethyl)-N N-dimethylammonium chloride, N N-(ditallowoxyl-oxy-ethyl)-N,N-dimethyl ammonium chloride, dialkyl imidazolium methyl sulfate, amido silicones, and mixtures thereof. The monoalky cationic active may be selected from cetyl trimethyl ammonium chloride, stearyl trimethyl ammonium chloride, and mixtures thereof.

The level of cationic active in a dilute fragrance delivery vehicle of the invention may be above 0.1%, for example above about 0.5%(wt), such as above about 1%(wt). In the present invention, the cationic active may be from about 0.5%(wt) to about 7.0%(wt), preferably about 0.7%(wt) to about 6.0%(wt), such as for example, about 1.0%(wt) to about 5.0%(wt). Levels of the cationic active in a concentrated delivery vehicle of the invention may be from about 7%(wt) to about 20%(wt), preferably about 10%(wt) to about 15%(wt).

The water insoluble oils of the fragrance delivery vehicle may be selected from, for example, mineral oils, ester oils, sugar ester oils or oily sugar derivatives, natural oils, such as vegetable oils, and mixtures thereof. Preferably, the water insoluble oil is a vegetable oil.

The water insoluble oils of the present invention are preferably hydrophilic with substantially no surface activity. The level of water insoluble oil in a dilute fragrance delivery vehicle of the invention is above about 0.1%(wt), such as for example, above about 0.5%(wt). Thus, the level of water insoluble oil in the fragrance delivery vehicle may be for example, from about 0.1%(wt) to about 7.0% (wt), preferably about 0.3%(wt) to about 6.0%(wt), such as for example, about 0.5%(wt) to about 5.0%(wt). In a concentrated fragrance delivery vehicle of the invention, the level of water insoluble oil is from about 1%(wt) to about 25%(wt), preferably about 3%(wt) to about 20%(wt). It is preferred that the water insoluble oil used in the present invention be in liquid form.

In the present invention, the terms “sugar ester oil,” “sucrose polyester” (SPE), and “oily sugar derivative” are disclosed in WO 00/7004, which is incorporated by reference as if recited in full herein. Preferably, the ester oils are esters containing straight or branched, saturated or unsaturated carboxylic acids. The ester oil or oily sugar derivative is a liquid or soft solid derivative of a cyclic polyol, of a reduced saccharide or mixtures thereof, the resulting derivatives resulting from 35 to 100% of the hydroxyl groups in the polyol or in the saccharide being esterified or etherified. In the present invention, the derivative has two or more ester or ether groups independently attached to a C₈-C₂₂ alkyl or alkenyl chain.

The oily sugar derivatives of the invention are also referred to herein as “derivative-CP” and “derivative-RS” depending upon whether the derivative is a product derived from a cyclic polyol or from a reduced saccharide starting material respectively. Preferably, the derivative-CP and derivative-RS contain about 35% (wt) of tri- or higher esters, e.g. at least about 40%. Preferably about 35% to about 85%(wt), most preferably about 40% to about 80%(wt), even more preferably about 45% to about 75%(wt), such as about 45% to about 70%(wt) of the hydroxyl groups in the cyclic polyol or in the reduced saccharide are esterified or etherified to produce the derivative-CP and derivative-RS respectively.

For the derivative-CP and derivative-RS, the tetra, penta, etc. prefixes indicate the average degrees of esterification or etherification. The compounds exist as a mixture of materials ranging from the monoester to the fully esterified ester. It is the average degree of esterification as determined by weight that is referred to herein. The derivative-CP and derivative-RS used herein do not have substantial crystalline character at 20° C. Instead, they are preferably in a liquid or soft solid state, as hereinbelow defined, at 20° C.

The starting cyclic polyol or reduced saccharide material is esterified or etherified with C₈-C₂₂ alkyl or alkenyl chains to the appropriate extent of esterification or etherification so that the derivatives are in the requisite liquid or soft solid state. These chains may contain unsaturation, branching or mixed chain lengths.

Typically the derivative-CP or derivative-RS has 3 or more, preferably 4 or more, for example 3 to 8, such as 3 to 5, ester or ether groups or mixtures thereof. It is preferred two or more of the ester or ether groups of the derivative-CP and derivative-RS are, independently of one another, attached to a C₈ to C₂₂ alkyl or alkenyl chain. The alkyl or alkenyl groups may be branched or linear carbon chains.

In the present invention, the terms derivative-CP and derivative-RS encompass all ether or ester derivatives of all forms of saccharides, which fall into the above definition. Examples of preferred saccharides for the derivative-CP and derivative-RS to be derived from are monosaccharides and disaccharides.

Examples of monosaccharides within the scope of the present invention include xylose, arabinose, galactose, fructose, sorbose, and glucose. Glucose is especially preferred. An example of a reduced saccharide is sorbitan. Examples of disaccharides include maltose, lactose, cellobiose, and sucrose. Sucrose is especially preferred.

If the derivative-CP is based on a disaccharide, it is preferred that the disaccharide has three or more ester or ether groups attached to it. Examples of such sugars include sucrose tri-, tetra-, and penta- esters. Where the cyclic polyol is a reducing sugar, it is advantageous if each ring of the derivative-CP has one ether group, preferably at the C₁ position. Suitable examples of such compounds include methyl glucose derivatives. Examples of suitable derivative-CPs include esters of alkyl(poly)glucosides, in particular alkyl glucoside esters having a degree of polymerization from 1 to 2.

The HLB of the derivative-CP and derivative-RS is typically between 1 and 3.

The derivative-CP and derivative-RS may have branched or linear, alkyl or alkenyl chains (with varying degrees of branching), mixed chain lengths, and/or unsaturation. Those having unsaturated and/or mixed alkyl chain lengths are preferred.

One or more of the alkyl or alkenyl chains (independently attached to the ester or ether groups) may contain at least one unsaturated bond.

For example, predominantly unsaturated fatty chains may be attached to the ester/ether groups. Such unsaturated fatty chains attached to the ester/ether groups may be derived from rape oil, cotton seed oil, soybean oil, oleic, tallow, palmitoleic, linoleic, erucic or other sources of unsaturated vegetable fatty acids.

The alkyl or alkenyl chains of the derivative-CP and derivative-RS are preferably predominantly unsaturated, for example sucrose tetratallowate, sucrose tetrarapeate, sucrose tetraoleate, sucrose tetraesters of soybean oil or cotton seed oil, cellobiose tetraoleate, sucrose trioleate, sucrose triapeate, sucrose pentaoleate, sucrose pentarapeate, sucrose hexaoleate, sucrose hexarapeate, sucrose triesters, pentaesters and hexaesters of soybean oil or cotton seed oil, glucose trioleate, glucose tetraoleate, xylose trioleate, or sucrose tetra-,tri-, penta- or hexa- esters with any mixture of predominantly unsaturated fatty acid chains.

Certain derivative-CPs and derivative-RSs within the scope of the present invention, however, may be based on alkyl or alkenyl chains derived from polyunsaturated fatty acid sources, e.g. sucrose tetralinoleate. It is preferred that most, if not all, of the polyunsaturation be removed by partial hydrogenation if such polyunsaturated fatty acid chains are used.

Oily sugar derivatives suitable for use in the present invention include sucrose pentalaurate, sucrose tetraoleate, sucrose pentaerucate, sucrose tetraerucate, sucrose, pentaoleate, and the like. Suitable materials include some of the Ryoto series available from Mitsubishi Kagaku Foods Corporation, such as for example, Ryoto ER290.

The liquid or soft solid derivative-CPs and derivative-RSs are characterized as materials having a solid:liquid ratio of between 50:50 and 0:100 at 20° C. as determined by T₂ relaxation time NMR, preferably between 43:57 and 0:100, most preferably between 40:60 and 0:100, such as, 20:80 and 0:100. The T₂ NMR relaxation time is commonly used for characterizing solid:liquid ratios in soft solid products such as fats and margarines. For the purpose of the present invention, any component of the NMR signal with a T₂ of less than 100 microseconds is considered to be a solid component and any component with T₂ greater than 100 microseconds is considered to be a liquid component.

The liquid or soft solid derivative-CP and derivative-RS can be prepared by a variety of methods well known to those skilled in the art. These methods include acylation of the cyclic polyol or of a reduced saccharide with an acid chloride; trans-esterification of the cyclic polyol or of a reduced saccharide material with short chain fatty acid esters in the presence of a basic catalyst (e.g. KOH); acylation of the cyclic polyol or of a reduced saccharide with an acid anhydride; and acylation of the cyclic polyol or of a reduced saccharide with a fatty acid. Typical preparations of these materials are disclosed in AU 14416/88 (Procter and Gamble).

Preferably, the water insoluble oils used in the present invention are hydrophobic. It is also preferred that the water insoluble oil be an ester oil such as a sugar ester oil or an oil with substantially no surface activity.

Suitable oils include those in the Sirius range of mineral oils (e.g., Silkolene). Suitable ester oils include the saturated ester oils (e.g., Unichema) and the unsaturated sugar ester oils (e.g., Mitsubishi Kagaku). It is preferred that the ester oils of the invention be hydrophobic. It is further preferred that the ester oil be saturated (i.e., hardened) in nature, unless it is a sugar ester oil or a plant derivative, in which case, unsaturation is preferred.

Suitable ester oils are the fatty ester of a mono or polyhydric alcohol having from 1 to about 24 carbon atoms in the hydrocarbon chain and mono or polycarboxylic acids having from 1 to about 24 carbon atoms in the hydrocarbon chain, with the proviso that the total number of carbon atoms in the ester oil is equal to or greater than 16 and that at least one of the hydrocarbon radicals in the ester oil has 12 or more carbon atoms. Ester oils most suitable for use in the present invention are the PRIOLUBES from Unichema. In particular, PRIOLUBE 1407, PRIOLUBE 1447, PRIOLUBE 1415, PRIOLUBE 1446, PRIOLUBE 1427, PRIOLUBE 1445, PRIOLUBE 2045, PRIOLUBE 3988, PRIOLUBE 3987, PRIOLUBE 2091, ESTOL 1545, and ESTOL 1527 are advantageously employed in the present invention. Of these, ESTOL 1545, which is a 2-ethyl hexyl stearate, is particularly useful. Suitable mineral oils include the Esso Marcol technical grade range of oils, such as the Silkolene medicinal Sirius range (e.g., M40, M70, and Ml 80). The molecular weight of the mineral oil is typically within the range 150 to 400.

It is preferred that the density of the mineral oil be from 0.80 to 0.90 g/cm², such as for example from 0.83 to 0.88 g/cm². The viscosity of the ester oil or mineral oil may be from 2 mPas to 400 mPas at a temperature of 25° C., preferably a viscosity from 2 mPas to 150 mPas, such as for example, a viscosity from 10 mPas to 100 mPas. The viscosity of the sugar ester oil should be below 50,000 mPas, preferably 5,000 mPas to 20,000 mPas, such as for example from 6,000 mPas to 20,000 mPas. All viscosities are measured at 25° C. It is further preferred that the refractive index of the oil be from 1.445 to 1.490, such as from 1.460 to 1.485.

The fragrance composition of the present invention may be composed of aroma chemicals selected from one or more of the following classes: alcohols, aldehydes, ketones, esters, acetals, oximes, nitrites, ethers, and essential oils.

The fragrance delivery vehicle may optionally contain, viscosity modifiers, antioxidants, deposition aids, UV absorbers, non ionics, zwiterionics, dye transfer ingredients, enzymes, antimicrobial agents, cationic agents, antistatic agents, dyes, fatty acids, emulsifiers, shape retention agents, anti-wrinkling agents, color care agents, bluing agents, optical brighteners, shine enhancers, preservatives, anti-corrosion agents, insect repellent agents, and mixtures thereof.

The fragrance delivery vehicle of the present invention (i.e., the water insoluble oil, cationic active, and fragrance composition) also serves to improve the fragrance perception of any consumer product to which it is added. Thus, the fragrance delivery vehicle not only provides enhanced fragrance and conditioning to a substrate-to-be-treated (e.g., hair or fabric), but also provides or improves the fragrance of the underlying consumer product. Thus, in the present invention, a fragrance-improving quantity of the fragrance delivery vehicle may be incorporated (i.e., mixed into, or combined with) any suitable consumer product.

Accordingly, another embodiment of the invention is a process for enhancing the fragrance of a consumer product. This process includes incorporating a fragrance improving quantity of the fragrance delivery vehicle of the present invention into a consumer product.

In the present invention, a “fragrance-improving quantity of the fragrance delivery vehicle” corresponds to the amount of the fragrance delivery vehicle required to condition and impart a fragrance to a substrate and to impart a fragrance to the consumer product. Thus, a fragrance-improving quantity of the fragrance delivery vehicle as used herein means from about 0.5 to about 25%(wt), preferably from about 1% to about 20%(wt), such as for example from about 2% to about 15%(wt) based on the weight of the consumer product.

As used herein, a consumer product includes any commercially available product to which the present fragrance delivery vehicles may be added, without significantly altering the underlying function of the consumer product. Thus, a consumer product includes for example, laundry detergents, fabric and hair conditioners, shampoos, hair creams, fabric softeners, fabric dewrinklers, cleansers, and the like.

Another embodiment of the invention is a process for conditioning and fragrancing a substrate and fragrancing a product. This process includes combining a fragrance delivery vehicle of the present invention with a product for conditioning and fragrancing a substrate. The fragrance delivery vehicle contains a water insoluble oil, a conditioner containing a cationic active at a level above about 0.5%(wt), and at least about 0.1%(wt) of a fragrance composition including a first aroma chemical with a clogP of 3.0 or less and a second aroma chemical having a clogP of at least 4.0. As noted above, the fragrance composition preferably contains mixtures of one or more of the first and second aroma chemicals. The product containing the fragrance delivery vehicle is then contacted with a substrate in an aqueous medium.

In the present invention, the product containing the fragrance delivery vehicle is contacted with the substrate using any conventional method, such as for example by adding the product to the rinse cycle of a conventional washing machine in the case of a fabric. The product is typically contacted with the substrate in an aqueous medium. In the present invention, the aqueous medium is water or a solution containing a substantial amount of water (e.g., greater than about 70%, preferably greater than about 90-95% water) that is suitable for fabric or hair washing.

Preferably, the substrate is dried after it is contacted with the fragrance delivery vehicle-containing product. In the present invention, the substrate may be a fabric or hair. Preferably, the product is a consumer product as set forth previously.

As used herein, the term “substrate” means hair or a fabric (natural, synthetic, or natural/synthetic blends) that is suitable for conventional washing and drying.

Another embodiment of the present invention is a process for designing a fragrance delivery vehicle that provides fragrance to a consumer product and that provides fragrance and conditioning to a dry substrate when treated with the fragrance delivery vehicle. This process includes selecting a first aroma chemical having a clogP of 3 or less, which first aroma chemical is further selected from the group consisting of aroma chemicals having a vapor pressure at 25° C. of greater than 0.07 mm Hg, aroma chemicals having a threshold value at 25° C. of less than 100, and aroma chemicals having a vapor pressure at 25° C. of greater than 0.07 mm Hg and a threshold value at 25° C. of less than 100. The first aroma chemical is then combined with a second aroma chemical having a clogP of at least 4.0 using any conventional method, such as mixing, to form a fragrance composition. As noted above, the fragrance composition preferably contains mixtures of one or more of the first and second aroma chemicals. At least about 0.1%(wt) of the fragrance composition is then incorporated with a water insoluble oil and a conditioner containing a cationic active at a level above about 0.5%(wt) to form the fragrance delivery vehicle.

The following examples are provided to further illustrate the processes and compositions of the present invention. These examples are illustrative only and are not intended to limit the scope of the invention in any way.

### EXAMPLES

In the examples that follow, all percent are %wt unless otherwise noted.

### Example 1

The following softener bases were prepared by mixing the ingredients at 50° C.:

| Ingredients | 12% HEQ | 12% HEQ/4% SPE |
|---|---|---|
| HEQ* | 12.00% | 12.00% |
| SPE** | — | 4.00% |
| Perfume Swan026KA | 0.95% | 0.95% |
| Water, preservative, dye | To 100.00% | To 100.0% |

Swan026KA

| | | | Vapor Pressure |
|---|---|---|---|
| Ingredient | % (w/w) | cLogP | (mm Hg @ 25° C.) |
| Heliotropine | 2.00 | 1.1 | 0.01 |
| Coumarin | 1.75 | 1.4 | 0.002 |
| Anisic aldehyde | 1.30 | 1.7 | 0.039 |
| Hedione | 4.60 | 2.5 | 0.001 |
| Diethyl phthalate | 9.75 | 2.7 | 0.002 |
| Methyl naphthyl ketone | 0.70 | 2.9 | 0.001 |
| Dihydro myrcenol | 1.50 | 3.0 | 0.166 |
| Dimethyl benzyl carbinyl | 1.20 | 3.0 | 0.014 |
| acetate | | | |
| Peonil | 10.6 | 3.2 | 0.001 |
| Linalool | 1.40 | 3.3 | 0.091 |
| Citronellol | 1.90 | 3.4 | 0.015 |
| Yara yara | 2.30 | 3.4 | 0.01 |
| Cyclamen aldehyde | 0.90 | 3.7 | 0.009 |
| Florhydral | 1.20 | 3.7 | 0.02 |
| Ethyl linalool | 3.80 | 3.8 | 0.005 |
| Beta ionone | 2.40 | 3.9 | 0.017 |
| Alpha damascone | 0.10 | 4.0 | 0.008 |
| Benzyl salicylate | 1.60 | 4.0 | 0.001 |
| Linayl acetate | 0.30 | 4.1 | 0.116 |
| Lilial | 10.50 | 4.1 | 0.004 |
| Gamma methyl ionone | 4.20 | 4.3 | 0.01 |
| Gamma terpinene | 0.10 | 4.4 | 1.05 |
| Ebanol | 1.50 | 4.4 | 0.002 |
| Limonene | 3.70 | 4.6 | 1.51 |
| Tetra hydro linalool | 3.00 | 4.8 | 0.003 |
| Hexyl salicylate | 6.90 | 4.9 | 0.001 |
| Hexyl Cinnamic Aldehyde | 11.40 | 4.9 | 0.001 |
| Aldehyde C12 MNA | 0.40 | 5.0 | 1.428 |
| Radjanol | 1.40 | 5.0 | 0.001 |
| Iso-E Super | 4.30 | 5.3 | 0.001 |
| Thibetolide | 3.30 | 5.4 | 0.001 |

Each base containing the fragrance was equilibrated for 24 hours at room temperature.

Terry Toweling test pieces were desized by washing three times in a liquid detergent (Purex HDL liquid) at 50° C. in a conventional consumer washing machine. The toweling was washed with water four times. The toweling test pieces were then tumble-dried in a conventional consumer dryer set at normal cycle.

The test pieces were then rinsed in the respective softener in a Terg-O-Tometer using tap water at 25° C. with a cloth to liquor ratio of 1:25, a product concentration of 0.6% and an agitation of 65 rpm for 5 min. The test pieces were spun dry to a constant weight. The test pieces were then line dried.

Each test piece was placed in a glass headspace (HS) collection vessel and 2 liters of headspace was collected on a Tenax trap. The fragrance ingredients were thermally desorbed into an Agilent 6890 capillary GC fitted with a high sensitivity Mass Selective Detector. The amount of fragrance determined in the headspace is shown below:

| | Quantity of fragrance in headspace |
|---|---|
| | Off_dry_towels |
| Softener Base | Nanograms/liter |
| 12% HEQ | 749.5 |
| 12% HEQ/4% SPE | 3099.9 |

As the data show, the cationic oil-based softener is clearly delivering much more fragrance to dry fabric than the softener containing the cationic HEQ alone.

Upon smelling each softener composition, it was noted that the fragrance of the HEQ/SPE softener was significantly less intense compared to HEQ softener.

### Example 2

The following fragrance accords were made:

Accord A

| | | Vapor Pressure | |
|---|---|---|---|
| Ingredient | ClogP | (mm. Hg @ 25° C.) | % (w/w) |
| Coumarin | 1.4 | 0.002 | 10 |
| Methyl jasmonate | 2.1 | 0.001 | 10 |
| Methyl cinnamate | 2.2 | 0.008 | 10 |
| Eugenyl acetate | 2.4 | 0.008 | 10 |
| Hedione | 2.5 | 0.001 | 10 |
| Dihydro eugenol | 2.7 | 0.006 | 10 |
| Methyl iso eugenol | 3.1 | 0.002 | 10 |
| Phenoxanol | 3.2 | 0.001 | 10 |
| Benzophenone | 3.2 | 0.001 | 10 |
| Dipropylene glycol | — | — | 10 |
| (DPG)-Solvent | | | |

Accord B

| | | Vapor Pressure | |
|---|---|---|---|
| Ingredient | ClogP | (mm Hg @ 25° C.) | % (w/w) |
| Lilial | 4.1 | 0.002 | 25 |
| Hexyl Salicylate | 4.89 | 0.0008 | 25 |
| Galaxolide (50% in DEP) | 6.0 | 0.0002 | 25 |
| Fixolide | 6.4 | 0.0001 | 25 |

The following softener systems were prepared:

| Ingredients | 12% HEQ | 12% HEQ/4% SPE |
|---|---|---|
| HEQ | 13.00% | 6.5% |
| SPE | — | 6.5% |
| Perfume | 1.0% | 1.0% |
| Water, preservative, dye | To 100.00% | To 100.0% |

Where the perfume in the ratios set forth below (F-F50) were combined with the softener system set forth above:

| | |
|---|---|
| F | 100% A Accord |
| F10 | 90% Accord A + 10% Accord B |
| F20 | 80% Accord A + 20% Accord B |
| F30 | 70% Accord A + 30% Accord B |
| F40 | 60% Accord A + 40% Accord B |
| F50 | 50% Accord A + 50% Accord B |

Desized Terry Toweling test cloths were pre-washed in 2.05 g of a liquid detergent (Purex Free detergent liquid) in tap water at 100° F. in a Terg-O-Tometer. The cloth to liquor ratio was 1:20. The towels were rinsed once in 2 liters of tap water. To the final rinse, 2.05 g of the respective softener system was added and agitated for 5 minutes. The toweling pieces were spun dry.

The test pieces were then line dried and an expert panel of 5 people was asked to assess them as to whether they perceived that the fabric piece was fragranced. The results obtained are presented in Table 2:

TABLE 2

| Product | Detected fragrance | Did not detect fragrance |
|---|---|---|
| F | 1 | 4 |
| F10 | 1 | 4 |
| F20 | 3 | 2 |
| F30 | 4 | 1 |
| F40 | 5 | 0 |
| F50 | 5 | 0 |

Thus, in the fragrance delivery vehicle containing a water-insoluble oil the fragrance composition must have at least 20% of aroma chemicals with a clogP of at least 4.0 in order to fragrance dry fabric.

### Example 3

The following fragrance delivery vehicle samples were prepared:

3A 13% HEQ+0.9% F50 (from Example 1)+0.1% DPG (control)

3B 6.5% HEQ+6.5% SPE+0.9% F50+0.1% DPG

3C 6.5% HEQ+6.5% SPE+0.9% F50+0.04% Ocimene+0.06% DPG

3D 6.5% HEQ+6.5% SPE+0.9% F50+0.04% Allyl caproate+0.06% DPG

3E 6.5% HEQ+6.5% SPE+0.9% F50+0.04% Cyclal C+0.06% DPG

3F 6.5% HEQ+6.5% SPE+0.9% F50+0.04% cis 3 hexyl acetate+0.06% DPG

3G 6.5% HEQ+6.5% SPE+0.9% F50+0.04% Cyclohexyl acetate+0.06% DPG

Where:

| | clogP | Vapor Pressure (mm Hg at 25° C.) |
|---|---|---|
| Ocimene | 3.7 | 1.559 |
| Allyl caproate | 3.2 | 0.680 |
| Cyclal C | 2.67 | 0.578 |
| Cis 3 Hexenyl acetate | 2.5 | 1.182 |
| Cyclohexyl acetate | 2.24 | 1.21 |

After combining the ingredients, the samples were allowed to equilibrate for 24 hours at room temperature. Samples 3B to 3G were compared in paired comparisons against 3A (control) for fragrance intensity. 5 expert panelists were used. The results are shown in Table 3 below:

TABLE 3

| Sample Comparison | Number Selecting |
|---|---|
| 3A v 3B | 5 v 0 |
| 3A v 3C | 5 v 0 |
| 3A v 3D | 3 v 2 |
| 3A v 3E | 1 v 4 |
| 3A v 3F | 0 v 5 |
| 3A v 3G | 0 v 5 |

As the data show, the oil-based fragrance delivery vehicles of the present invention have a fragrance intensity that is at least equal to the non-oil containing base when the fragrance composition contains an aroma chemical with a clogP below 3.0 (e.g., Samples 3E, 3F, and 3G).

### Example 4

The following fragrance delivery vehicle samples were prepared:

4A 13% HEQ+0.9% F50+0.1% DPG (control)

4B 6.5% HEQ+6.5% SPE+0.9% F50+0.1I% DPG

4C 6.5% HEQ+6.5% SPE+0.9% F50+0.04% Phenyl propyl alcohol+0.06% DPG

4D 6.5% HEQ+6.5% SPE+0.9% F50+0.04% Phenyl ethyl alcohol+0.06% DPG

4E 6.5% HEQ+6.5% SPE+0.9% F50+0.04% Benzyl acetate+0.06% DPG

4F 6.5% HEQ+6.5% SPE+0.9% F50+0.04% Benzyl formate+0.06% DPG

4G 6.5% HEQ+6.5% SPE+0.9% F50+0.04% Anapear+0.06% DPG

4H 6.5% HEQ+6.5% SPE+0.9% F50+0.04% Alcohol C6+0.06% DPG

4I 6.5% HEQ+6.5% SPE+0.9% F50+0.04% cis-3-hexenol+0.06% DPG

4J 6.5% HEQ+6.5% SPE+0.9% F50+0.04% trans-2-hexenal+0.06% DPG

Where the fragrance ingredient in the respective perfume compositions was:

| Fragrance Ingredient | Clog P | Vapor Pressure (mm Hg at 25° C.) |
|---|---|---|
| Phenyl propyl alcohol | 1.9 | 0.027 |
| Phenyl ethyl alcohol | 1.4 | 0.074 |
| Benzyl acetate | 1.9 | 0.164 |
| Benzyl formate | 1.5 | 0.270 |
| Anapear | 1.6 | 0.777 |
| Alcohol C6 | 1.9 | 0.947 |
| cis-3-hexenol | 1.6 | 1.040 |
| trans-2-hexenal | 1.6 | 11.200 |

After mixing the ingredients, the fragrance delivery samples were allowed to equilibrate for 24 hours at room temperature. Samples 4B to 4J were compared in paired comparisons against 4A (control) for fragrance intensity. 5 expert panelists were used. The results are shown in Table 4 below:

TABLE 4

| Sample Comparison | Number Selecting |
|---|---|
| 4A v 4B | 5 v 0 |
| 4A v 4C | 4 v 1 |
| 4A v 4D | 3 v 2 |
| 4A v 4E | 1 v 4 |
| 4A v 4F | 1 v 4 |
| 4A v 4G | 1 v 4 |
| 4A v 4H | 1 v 4 |
| 4A v 4I | 0 v 5 |
| 4A v 4J | 0 v 5 |

As the data demonstrate, the intensity of the fragrance is maintained in the fragrance delivery vehicle with aroma chemicals with a vapor pressure above 0.07 mm Hg vapor pressure at 25° C., preferably greater than 0.1 mm Hg vapor pressure at 25 C.

### Example 5

Low Odor Intensity Materials:

Phenyl ethyl alcohol and Fructone are relatively low odor intensity materials:

| Ingredient | clogP | Vapor Pressure (mm Hg at 25° C.) |
|---|---|---|
| Phenyl ethyl alcohol | 1.4 | 0.074 |
| Fructone | 0.6 | 0.086 |

The following fragrance delivery samples were prepared:

6A 13% HEQ+0.9% F50+0.1% DPG (control)

6B 6.5% HEQ+6.5% SPE+0.9% F50+0.1% DPG

6C 6.5% HEQ+6.5% SPE=0.9% F50+0.02% Phenyl ethyl alcohol+0.08% DPG

6D 6.5% HEQ+6.5% SPE=0.9% F50+0.04% Phenyl ethyl alcohol+0.06% DPG

6E 6.5% HEQ+6.5% SPE=0.9% F50+0.06% Phenyl ethyl alcohol+0.04% DPG

6F 6.5% HEQ+6.5% SPE=0.9% F50+0.08% Phenyl ethyl alcohol+0.02% DPG

6G 6.5% HEQ+6.5% SPE=0.9% F50+0.1% Phenyl ethyl alcohol

After mixing, the fragrance delivery samples were allowed to equilibrate for 24 hours at room temperature. Samples 6B to 6G were compared in paired comparisons against 6A (control) for fragrance intensity. 5 expert panelists were used. The results are shown in Table 5 below:

TABLE 5

| Sample Comparison | Number Selecting |
|---|---|
| 6A v 6B | 5 v 0 |
| 6A v 6C | 4 v 1 |
| 6A v 6D | 3 v 2 |
| 6A v 6E | 1 v 4 |
| 6A v 6F | 1 v 4 |
| 6A v 6G | 0 v 5 |

The following fragrance delivery vehicle samples were prepared:

6H 6.5% HEQ+6.5% SPE=0.9% F50+0.02% Fructone+0.08% DPG

6I 6.5% HEQ+6.5% SPE=0.9% F50+0.04% Fructone+0.06% DPG

6J 6.5% HEQ+6.5% SPE=0.9% F50+0.06% Fructone+0.04% DPG

6K 6.5% HEQ+6.5% SPE=0.9% F50+0.08% Fructone+0.02% DPG

6L 6.5% HEQ+6.5% SPE=0.9% F50+0.1% Fructone

After mixing, the fragrance delivery vehicle samples were allowed to equilibrate for 24 hours at room temperature. Samples 6H to 6L were compared in paired comparisons against 6A (control) for fragrance intensity. 5 expert panelists were used. The results are shown in Table 6 below:

TABLE 6

| Sample Comparison | Number Selecting |
|---|---|
| 6A v 6H | 3 v 2 |
| 6A v 6I | 2 v 3 |
| 6A v 6J | 1 v 4 |
| 6A v 6K | 1 v 4 |
| 6A v 6L | 0 v 5 |

As the data indicate, low odor intensity materials when used alone with the F50 fragrance must account for at least 2% of the fragrance to maintain the intensity that the fragrance would have in a non-oil based cationic system.

### Example 6

Moderate Odor Intensity Materials

The following fragrance ingredients a have a moderate odor intensity.

| Ingredient | clogP | Vapor Pressure (mm Hg at 25° C.) |
|---|---|---|
| Methyl amyl ketone | 2.0 | 4.732 |
| Phenyl acetate | 2.1 | 3.987 |
| Cyclohexyl acetate | 2.2 | 0.978 |
| Aldehyde C7 | 2.5 | 3.854 |
| Aldehyde C8 | 3.0 | 1.409 |

The following fragrance delivery vehicle samples were prepared:

7A 13% HEQ+0.9% F50+0.1% DPG (control)

7B 6.5% HEQ+6.5% SPE=0.9% F50+0.004% Methyl amyl ketone+0.096% DPG

7C 6.5% HEQ+6.5% SPE=0.9% F50+0.006% Methyl amyl ketone+0.094% DPG

7D 6.5% HEQ+6.5% SPE=0.9% F50+0.008% Methyl amyl ketone+0.092% DPG

7E 6.5% HEQ+6.5% SPE=0.9% F50+0.01% Methyl amyl ketone+0.09% DPG

7F 6.5% HEQ+6.5% SPE=0.9% F50+0.02% Methyl amyl ketone+0.08% DPG

After mixing, the fragrance delivery vehicle samples were allowed to equilibrate for 24 hours at room temperature. Samples 7B to 7F were compared in paired comparisons against 7A (control) for fragrance intensity. 5 expert panelists were used. The results are shown in Table 7 below:

TABLE 7

| Sample Comparison | Number Selecting |
|---|---|
| 7A v 7B | 4 v 1 |
| 7A v 7C | 4 v 1 |
| 7A v 7D | 1 v 4 |
| 7A v 7E | 0 v 5 |
| 7A v 7F | 0 v 5 |

The following fragrance delivery samples were prepared:

7G 6.5% HEQ+6.5% SPE=0.9% F50+0.002% Prenyl acetate+0.098% DPG

7H 6.5% HEQ+6.5% SPE=0.9% F50+0.004% Prenyl acetate+0.096% DPG

7I 6.5% HEQ+6.5% SPE=0.9% F50+0.006% Prenyl acetate +0.094% DPG

7J 6.5% HEQ+6.5% SPE=0.9% F50+0.008% Prenyl acetate+0.092% DPG

7K 6.5% HEQ+6.5% SPE=0.9% F50+0.01% Prenyl acetate

After mixing, the fragrance delivery samples were allowed to equilibrate for 24 hours at room temperature. Samples 7G to 7K were compared in paired comparisons against 7A (control) for fragrance intensity. 5 expert panelists were used. The results are shown in Table 8 below:

TABLE 8

| Sample Comparison | Number Selecting |
|---|---|
| 7A v 7G | 2 v 3 |
| 7A v 7H | 1 v 4 |
| 7A v 7I | 0 v 5 |
| 7A v 7J | 0 v 5 |
| 7A v 7K | 0 v 5 |

The following fragrance delivery vehicle samples were prepared:

7L 6.5% HEQ+6.5% SPE=0.9% F50+0.0003% Cyclohexyl acetate+0.0997% DPG

7M 6.5% HEQ+6.5% SPE=0.9% F50+0.0006% Cyclohexyl acetate+0.0994% DPG

7N 6.5% HEQ+6.5% SPE 0.9% F50+0.001% Cyclohexyl acetate+0.099% DPG

After mixing, the fragrance delivery samples were allowed to equilibrate for 24 hours at room temperature. Samples 7L to 7N were compared in paired comparisons against 7A (control) for fragrance intensity. 5 expert panelists were used. The results are shown in Table 9 below:

TABLE 9

| Sample Comparison | Number Selecting |
|---|---|
| 7A v 7L | 2 v 3 |
| 7A v 7M | 0 v 5 |
| 7A v 7N | 0 v 5 |

The following fragrance delivery vehicle samples were prepared:

7O 6.5% HEQ+6.5% SPE=0.9% F50+0.0001% Aldehyde C7+0.0999% DPG

7P 6.5% HEQ+6.5% SPE=0.9% F50+0.0003% Aldehyde C7+0.0997% DPG

After mixing, the fragrance delivery samples were allowed to equilibrate for 24 hours at room temperature. Samples 70 to 7P were compared in paired comparisons against 7A (control) for fragrance intensity. 5 expert panelists were used. The results are shown in Table 10 below:

TABLE 10

| Sample Comparison | Number Selecting |
|---|---|
| 7A v 7O | 1 v 4 |
| 7A v 7P | 0 v 5 |

The following fragrance delivery vehicles were prepared:

7Q 6.5% HEQ+6.5% SPE=0.9% F50+0.0001% Aldehyde C8+0.0999% DPG

7R 6.5% HEQ+6.5% SPE=0.9% F50+0.0003% Aldehyde C8+0.0997% DPG

After mixing, the fragrance delivery samples were allowed to equilibrate for 24 hours at room temperature. Samples 7Q to 7R were compared in paired comparisons against 7A (control) for fragrance intensity. 5 expert panelists were used. The results are shown in Table 11 below:

TABLE 11

| Sample Comparison | Number Selecting |
|---|---|
| 7A v 7Q | 1 v 4 |
| 7A v 7R | 0 v 5 |

As the data indicate, selecting moderate odor intensity aroma chemicals, having a clogP of 3.0 or less and a vapor pressure in excess of 0.07 mm Hg at 25° C., when incorporated into a fragrance above 0.05% is sufficient to maintain good fragrance intensity in a cationic-oil system.

### Example 7

High Odor Intensity Materials

The following materials have high odor intensity:

| Ingredient | clogP | Vapor Pressure (mm Hg @ 25° C.) |
|---|---|---|
| Ethyl-2-methyl-butyrate | 1.6 | 7.853 |
| Manzanate | 2.7 | 2.908 |

The following fragrance delivery vehicles were prepared:

8A 6.5% HEQ+6.5% SPE 0.9% F50+0.0001% Ethyl-2-methyl-butyrate+0.0999% DPG

8B 6.5% HEQ+6.5% SPE=0.9% F50+0.0003% Ethyl-2-methyl-butyrate+0.0997% DPG

After mixing, the fragrance delivery samples were allowed to equilibrate for 24 hours at room temperature. Samples 8A to 8B were compared in paired comparisons against 7A (control) for fragrance intensity. 5 expert panelists were used. The results are shown in Table 11 below:

TABLE 11

| Sample Comparison | Number Selecting |
|---|---|
| 7A v 8A | 1 v 4 |
| 7A v 8B | 0 v 5 |

The following fragrance delivery vehicle samples were prepared:

8C 6.5% HEQ+6.5% SPE=0.9% F50+0.0001% Manzanate+0.0999% DPG

8D 6.5% HEQ+6.5% SPE=0.9% F50+0.0003% Manzanate+0.0997% DPG

After mixing, the fragrance delivery samples were allowed to equilibrate for 24 hours at room temperature. Samples 8C to 8D were compared in paired comparisons against the control (7A) for fragrance intensity. 5 expert panelists were used. The results are shown in Table 12 below:

TABLE 12

| Sample Comparison | Number Selecting |
|---|---|
| 7A v 8C | 1 v 4 |
| 7A v 8D | 0 v 5 |

As the data indicate, high intensity aroma chemicals having a clogp of 3.0 or less and a vapor pressure in excess of 0.07 mm Hg at 25 C, when incorporated into a fragrance above 0.01% is sufficient to maintain good fragrance intensity in a cationic-oil system.

### Example 8

The following products were prepared:

| Ingredient | Dilute | Concentrated |
|---|---|---|
| HEQ | 5.0 | 12 |
| SPE | 1.7 | 4.0 |
| Coconut 3 EO non-ionic | — | 0.7 |
| Cetyl hydroxy ethyl | 0.03 | — |
| cellulose | | |
| Proxel (preservative) | 0.16 | 0.15 |
| Dye | 0.0015 | 0.0048 |
| Pearleacer(mica) | 0.1 | 0.0048 |
| Perfume* | 0.32 | 0.95 |
| Water | To 100 | To 100 |

*The following fragrances were used in the perfume:
Dilute: Either Swan026KA (see formulation in Example 1) or Swan026KB (see formulation below).
Concentrated: Either SwanK026KA or Swan026KB.

Dilute: Either Swan026KA (see formulation in Example 1) or Swan026KB (see formulation below).

Concentrated: Either SwanK026KA or Swan026KB.

Swan026KB

| | | | Vapor Pressure |
|---|---|---|---|
| Ingredient | % (w/w) | CLogP | (mm Hg @ 25° C.) |
| Coumarin | 1.75 | 1.4 | 0.002 |
| Ethyl methyl butyrate | 0.05 | 1.6 | 7.853 |
| Anapear | 0.85 | 1.6 | 0.777 |
| Acetophenone | 0.50 | 1.7 | 0.851 |
| Anisic aldehyde | 1.30 | 1.7 | 0.039 |
| Hedione | 4.60 | 2.5 | 0.001 |
| Cyclal C | 0.60 | 2.7 | 0.578 |
| Diethylphthalate | 9.75 | 2.7 | 0.002 |
| Methyl naphthyl ketone | 0.70 | 2.9 | 0.001 |
| Dihydro myrcenol | 1.50 | 3.0 | 0.166 |
| Dimethyl benzyl carbinyl | 1.20 | 3.0 | 0.014 |
| acetate | | | |
| Peonil | 10.6 | 3.2 | 0.001 |
| Linalool | 1.40 | 3.3 | 0.091 |
| Citronellol | 1.90 | 3.4 | 0.015 |
| Yara yara | 2.30 | 3.4 | 0.01 |
| Cyclamen aldehyde | 0.90 | 3.7 | 0.009 |
| Florhydral | 1.20 | 3.7 | 0.02 |
| Ethyl linalool | 3.80 | 3.8 | 0.005 |
| Beta ionone | 2.40 | 3.9 | 0.017 |
| Alpha damascone | 0.10 | 4.0 | 0.008 |
| Benzyl salicylate | 1.60 | 4.0 | 0.001 |
| Linayl acetate | 0.30 | 4.1 | 0.116 |
| Lilial | 10.50 | 4.1 | 0.004 |
| Gamma methyl ionone | 4.20 | 4.3 | 0.01 |
| Gamma terpinene | 0.10 | 4.4 | 1.05 |
| Ebanol | 1.50 | 4.4 | 0.002 |
| Limonene | 3.70 | 4.6 | 1.51 |
| Tetra hydro linalool | 3.00 | 4.8 | 0.003 |
| Hexyl salicylate | 6.90 | 4.9 | 0.001 |
| Hexyl Cinnamic Aldehyde | 11.40 | 4.9 | 0.001 |
| Aldehyde C12 MNA | 0.40 | 5.0 | 1.428 |
| Radjanol | 1.40 | 5.0 | 0.001 |
| Iso-E Super | 4.30 | 5.3 | 0.001 |
| Thibetolide | 3.30 | 5.4 | 0.001 |

After mixing, the products were allowed to equilibrate for 24 hours at room temperature. The products were rated for fragrance intensity. 5 expert panelists were used. The results are shown in Table 13 below:

TABLE 13

Number selecting

| Product Comparison | Swan 026 KA | Swan 026 KB |
|---|---|---|
| Dilute | 0 | 5 |
| Concentrated | 0 | 5 |

### Example 9

The following bases were prepared:

| | | Arquad/Semtol (cationic |
|---|---|---|
| Ingredient | Arquad (cationic alone) | and oil) |
| Arquad 2HT | 3.80 | 2.2 |
| (75% ai) | | |
| Semtol 70/28 | — | 3.3 |
| Pristerine 4981 | 0.63 | 0.37 |
| Cetyl hydroxyethyl | 0.03 | 0.03 |
| Perfume* | 0.25 | 0.25 |
| Dye | 0.1 | 0.1 |
| Formalin(36%) | 0.08 | 0.08 |
| Water | To 100 | To 100 |

* The following fragrances were used in the perfume:

Arquad: Swan026KA

Arquad/Semtol: Either Swan 026KA or Swan 026KB.

After mixing, the products were allowed to equilibrate for 24 hours at room temperature. A comparison between the Arquad product containing the Swan 026KA fragrance and the Arquad/Semtol product containing the Swan 026KA fragrance was made. In addition, a comparison of the Arquad product containing the Swan 026KA fragrance and the Arquad/Semtol product containing the Swan 026KB fragrance was made. The products were rated for fragrance intensity. 5 expert panelists were used. The results are shown in Table 14 below:

TABLE 14

| Product Comparison | Number selecting |
|---|---|
| Arquad Swan 026 KA v Arquad/Semtol | 5 v 0 |
| Swan 026 KA | |
| Arquad Swan 026 KA v Arquad/Semtol | 2 v 3 |
| Swan 026 KB | |

The invention being thus described, it will be obvious that the same may be varied in many ways. Such variations are not to be regarded as a departure from the spirit and scope of the invention and all such modifications are intended to be included within the scope of the following claims.

What is claimed is:

1. A fragrance delivery vehicle comprising a water insoluble oil, a conditioner containing a cationic active at a level above about 0.5%(wt), and at least about 0.1%(wt) of a fragrance composition comprising a first aroma chemical with a clogp of 3.0 or less and a second aroma chemical having a clogp of at least 4.0.

2. A fragrance delivery vehicle according to claim 1, wherein the first aroma chemical has a clogP of 2.5 or less.

3. A fragrance delivery vehicle according to claim 1, wherein the fragrance composition comprises a mixture of aroma chemicals having a clogP of 3 or less and an aroma chemical having a clogP of at least 4, an aroma chemical having a clogP of 3 or less and a mixture of aroma chemicals having a clogP of at least 4, and mixtures of aroma chemicals having a clogP of 3 or less and a clogP of at least 4.

4. A fragrance delivery vehicle according to claim 1, wherein the first aroma chemical is present in the fragrance delivery vehicle in a quantity that is at least about 0.5%(wt) based on the total weight of the fragrance composition.

5. A fragrance delivery vehicle according to claim 4, wherein the first aroma chemical is present in the fragrance delivery vehicle in a quantity that is at least about 1.0%(wt) based on the total weight of the fragrance composition.

6. A fragrance delivery vehicle according to claim 1, wherein the first aroma chemical has a vapor pressure at 25° C. of greater than about 0.07 mm Hg.

7. A fragrance delivery vehicle according to claim 6, wherein the first aroma chemical has a vapor pressure at 25° C. of greater than about 0.7 mm Hg.

8. A fragrance delivery vehicle according to claim 7, wherein the first aroma chemical has a vapor pressure at 25° C. of greater than about 1.0 mm Hg.

9. A fragrance delivery vehicle according to claim 1, wherein the first aroma chemical has a vapor pressure at 25° C. of between about 0.07 mm Hg and about 20.00.

10. A fragrance delivery vehicle according to claim 1, wherein the second aroma chemical is present in the fragrance delivery vehicle in a quantity that is at least about 20%(wt) based on the total weight of the fragrance composition.

11. A fragrance delivery vehicle according to claim 1, wherein the second aroma chemical has a vapor pressure at 25° C. of less than about 0.02 mm Hg.

12. A fragrance delivery vehicle according to claim 11, wherein the second aroma chemical has a vapor pressure at 25° C. of less than about 0.01 mm Hg.

13. A fragrance delivery vehicle according to claim 12, wherein the second aroma chemical has a vapor pressure at 25° C. of less than about 0.008 mm Hg.

14. A fragrance delivery vehicle according to claim 1, wherein the first aroma chemical has a perception threshold below 100 ng per liter at 25° C.

15. A fragrance delivery vehicle according to claim 14, wherein the first aroma chemical has a perception threshold below 50 ng per liter at 25° C.

16. A fragrance delivery vehicle according to claim 15, wherein the first aroma chemical has a perception threshold of below 30 ng per liter at 25° C.

17. A fragrance delivery vehicle according to claim 1, wherein the cationic active is selected from the group consisting of a dialky cationic active, a mono alky cationic active, and mixtures thereof.

18. A fragrance delivery vehicle according to claim 17, wherein the dialky cationic active is selected from the group consisting of dialkyldimethyl ammonium chloride, dialkyldimethyl ammonium methyl sulfate, di (hydrogenated tallow) dimethyl ammonium chloride, dihexadecyldiethylammonium chloride, distearyldimethylammonium chloride, dibehenyldimethylammoinium chloride, di(coconut alkyl)dimethyl ammonium chloride, ditallowdimethyl ammonium chloride, ester quatemium compounds, dialkylyloxy dimethyl ammonium chloride, N,N-di(tallowyl-oxy-ethyl)-N N-dimethylammonium chloride, N N-(ditallowoxyl-oxy-ethyl)-N,N-dimethyl ammonium chloride, dialkyl imidazolium methyl sulfate, amido silicones, and mixtures thereof.

19. A fragrance delivery vehicle according to claim 17, wherein the monoalky cationic active is selected from the group consisting of cetyl trimethyl ammonium chloride, stearyl trimethyl ammonium chloride, and mixtures thereof.

20. A fragrance delivery vehicle according to claim 1, wherein the water insoluble oil is selected from the group consisting of mineral oils, ester oils, sugar ester oils, natural oils, and mixtures thereof.

21. A fragrance delivery vehicle according to claim 1, wherein the water insoluble oils are hydrophobic with substantially no surface activity.

22. A fragrance delivery vehicle according to claim 20, wherein the natural oil is a vegetable oil.

23. A fragrance delivery vehicle according to claim 20, wherein the sugar ester oil is a sucrose polyester.

24. A fragrance delivery vehicle according to claim 20, wherein the sugar ester oil is selected from the group consisting of an oily sugar derived from a cyclic polyol (derivative-CP), an oily sugar derived from a reduced saccharide (derivative-RS), and mixtures thereof.

25. A fragrance delivery vehicle according to claim 1, wherein the fragrance composition is at least about 0.2%(wt) based on the weight of the fragrance delivery vehicle.

26. A fragrance delivery vehicle according to claim 1, wherein the fragrance composition is at least about 0.3%(wt) based on the weight of the fragrance delivery vehicle.

27. A fragrance delivery vehicle according to claim 1, wherein the fragrance composition is between about 0.1%(wt) to about 2.0%(wt) based on the weight of the fragrance delivery vehicle.

28. A fragrance delivery vehicle according to claim 1, wherein the first aroma chemical is present in the fragrance composition at a level that is at least 1% (wt) based on the total weight of the fragrance composition.

29. A fragrance delivery vehicle according to claim 28, wherein the first aroma chemical is present in the fragrance composition at a level that is at least 5% (wt) based on the total weight of the fragrance composition.

30. A fragrance delivery vehicle according to claim 1, wherein the first aroma chemical is present in the fragrance composition at a level that is between about 10-20% (wt) based on the total weight of the fragrance composition.

31. A process for conditioning and fragrancing a substrate and fragrancing a product comprising:
(a) combining a fragrance delivery vehicle with a product for conditioning and fragrancing a substrate, the fragrance delivery vehicle comprising a water insoluble oil, a conditioner containing a cationic active at a level above about 0.5%(wt), and at least about 0.1%(wt) of a fragrance composition comprising a first aroma chemical with a clogP of 3.0 or less and a second aroma chemical having a clogP of at least 4.0; and
(b) contacting the product containing the fragrance delivery vehicle with a substrate in an aqueous medium.

32. A process according to claim 31, further comprising drying the substrate.

33. A process according to claim 32, wherein the dry substrate is a fabric or hair.

34. A process according to claim 31, wherein the product is selected from the group consisting of laundry detergents, fabric conditioners, hair conditioners, shampoos, hair creams, fabric softeners, fabric dewrinklers, and cleansers.

35. A process according to claim 31, wherein the fragrance composition comprises a mixture of aroma chemicals having a clogp of 3 or less and an aroma chemical having a clogp of at least 4, an aroma chemical having a clogp of 3 or less and a mixture of aroma chemicals having a clogp of at least 4, and mixtures of aroma chemicals having a clogp of 3 or less and a clogp of at least 4.

36. A process for enhancing the fragrance of a consumer product comprising incorporating a fragrance improving quantity of a fragrance delivery vehicle according to claim 1 into a consumer product.

37. A process according to claim 36, wherein the consumer product is selected from the group consisting of laundry detergents, fabric conditioners, hair conditioners, shampoos, hair creams, fabric softeners, fabric dewrinklers, and cleansers.

38. A process according to claim 36, wherein the fragrance delivery vehicle comprises from about 0.5%(wt) to about 25%(wt) of the product.

39. A process according to claim 38, wherein the fragrance delivery vehicle comprises from about 1%(wt) to about 20%(wt) of the product.

40. A process according to claim 39, wherein the fragrance delivery vehicle comprises from about 2%(wt) to about 15%(wt) of the product.

41. A process according to claim 36, wherein the fragrance composition comprises a mixture of aroma chemicals having a clogp of 3 or less and an aroma chemical having a clogp of at least 4, an aroma chemical having a clogP of 3 or less and a mixture of aroma chemicals having a clogP of at least 4, and mixtures of aroma chemicals having a clogP of 3 or less and a clogP of at least 4.

42. A process for designing a fragrance delivery vehicle that provides fragrance to a consumer product and that provides fragrance and conditioning to a dry substrate, which process comprises:
(a) selecting a first aroma chemical having a clogP of 3 or less, which first aroma chemical is further selected from the group consisting of aroma chemicals having a vapor pressure at 25° C. of greater than 0.07 mm Hg, aroma chemicals having a threshold value at 25° C. of less than 100, and aroma chemicals having a vapor pressure at 25° C. of greater than 0.07 mm Hg and a threshold value at 25° C. of less than 100;
(b) combining the first aroma chemical with a second aroma chemical having a clogp of at least 4.0 to form a fragrance composition; and
(c) incorporating at least about 0.1%(wt) of the fragrance composition with a water insoluble oil and a conditioner containing a cationic active at a level above about 0.5%(wt) to form the fragrance delivery vehicle.

43. A process according to claim 42, wherein the fragrance composition comprises a mixture of aroma chemicals having a clogP of 3 or less and an aroma chemical having a clogP of at least 4, an aroma chemical having a clogP of 3 or less and a mixture of aroma chemicals having a clogP of at least 4, and mixtures of aroma chemicals having a clogP of 3 or less and a clogP of at least 4.
